(19) United States
(12) Patent Application Publication
(10) Pub. No.: US 2025/0346861 A1
(43) Pub. Date: Nov. 13, 2025

(54) SYSTEMS AND METHODS FOR GENERATING PLATELET LYSATE FROM PLATELET-RICH PLASMA

(71) Applicant: David Keith Harris, Austin, TX (US)

(72) Inventor: David Keith Harris, Austin, TX (US)

(21) Appl. No.: 18/804,898

(22) Filed: Aug. 14, 2024

Related U.S. Application Data
(60) Provisional application No. 63/645,415, filed on May 10, 2024.

(51) Int. Cl. C12N 5/078; C12M 1/26; C12M 1/34; C12M 1/42; C12N 13/00

(52) U.S. Cl. CPC C12N 5/0644; C12M 33/14; C12M 35/04; C12M 41/12; C12N 13/00

(57) ABSTRACT

A method for generating platelet lysate from platelet-rich plasma (PRP) includes placing a predetermined volume of PRP into a centrifuge tube. The method further includes sealing the centrifuge tube and placing the sealed centrifuge tube containing the PRP into a water bath. The method further includes operating an ultrasonic generator to direct ultrasonic waves onto the PRP in the sealed centrifuge tube in the water bath for a predetermined amount of time, thereby generating platelet lysate from the PRP. The method further includes spinning the sealed centrifuge tube containing the platelet lysate in a centrifuge, thereby separating byproducts out of the platelet lysate.

### CROSS-REFERENCE TO RELATED APPLICATIONS

This application claims the benefit of U.S. Provisional Application No. 63/645,415, filed May 10, 2024, which is incorporated herein by reference in its entirety.

### TECHNICAL FIELD

This disclosure generally relates to platelet lysate, and more specifically to systems and methods for generating platelet lysate from platelet-rich plasma (PRP).

### BACKGROUND

Platelet lysate is the product of platelet activation to obtain the desired growth factors contained within the platelets. Platelet lysate may be used to stimulate tissue and wound healing, and to separate out the relatively inflammatory platelet cellular membrane proteins from the more clinically-desirable anti-inflammatory and growth-promoting proteins that facilitate healing. This release of platelet vesicles and granules has many uses in medicine, including stem cell culture media, tissue stimulation for healing, stimulation to promote nerve healing, scar tissue modification and reduction, and joint injection to reduce inflammation. The release of platelet vesicles and granules further promotes a more favorable environment for cartilage repair and enzymatic reduction of unhealthy tissue mass such as in spinal disk herniation, scar, and de-vascularized tissue that is associated with injury. Producing platelet lysate, however, is typically a time consuming and inefficient process.

### SUMMARY

The present disclosure achieves technical advantages as systems, methods, and computer-readable storage media for generating platelet lysate from platelet-rich plasma (PRP). The present disclosure provides for a system integrated into a practical application with meaningful limitations that may include electronically communicating one or more first commands to a chilling system, the one or more first commands operable to control the chilling system to chill a water bath to a predetermined temperature according to a plurality of platelet lysate parameters. Other meaningful limitations of the system integrated into a practical application include: electronically communicating one or more second commands to an ultrasonic generator, the one or more second commands operable to control the ultrasonic generator to direct ultrasonic waves onto the PRP in the sealed centrifuge tube in the water bath for a predetermined amount of time according to the plurality of platelet lysate parameters, thereby generating platelet lysate from the PRP; and electronically communicating one or more third commands to a centrifuge, the one or more third commands operable to control the centrifuge to spin the sealed centrifuge tube containing the platelet lysate in a centrifuge at a spin rate according to the plurality of platelet lysate parameters, thereby separating byproducts out of the platelet lysate.

The present disclosure solves the technological problem of a lack of an efficient and effective process for generating platelet lysate from PRP. The technological solutions provided herein, and missing from conventional systems, are more than a mere application of a manual process to a computerized environment, but rather include functionality to implement a technical process to supplement current manual solutions for generating platelet lysate from PRP. In doing so, the present disclosure goes well beyond a mere application the manual process to a computer.

Unlike existing solutions that are inefficient and time consuming, embodiments of this disclosure provide systems and methods that provide functionality for optimally generating platelet lysate from PRP. By providing optimized generation of platelet lysate from PRP, the efficiency and effectiveness of certain outpatient procedures may be increased. For example, the time required to generate platelet lysate from PRP may be greatly decreased and the amount of platelet lysate generated from PRP may be greatly increased. Other technical advantages will be readily apparent to one skilled in the art from the following figures, descriptions, and claims. Moreover, while specific advantages have been enumerated above, various embodiments may include all, some, or none of the enumerated advantages.

In some embodiments, the disclosed embodiments utilize models that are formulated or otherwise configured to utilize various constraints and objectives in order to perform or execute a designated task (e.g., one or more features for generating platelet lysate from PRP, in accordance with one or more embodiments of the present disclosure). In other embodiments, the present disclosure includes techniques for implementing and training models (e.g., machine-learning models, artificial intelligence models, algorithmic constructs, optimizers, etc.) for performing or executing a designated task or a series of tasks (e.g., one or more features for generating platelet lysate from PRP, in accordance with one or more embodiments of the present disclosure). In these embodiments, the disclosed techniques provide a systematic approach for the training of such models to enhance performance, accuracy, and efficiency in their respective applications. In embodiments, the techniques for training the models can include collecting a set of data from a database, conditioning the set of data to generate a set of conditioned data, and/or generating a set of training data including the collected set of data and/or the conditioned set of data.

In some embodiments, a model can undergo a training phase wherein the model may be exposed to the set of training data, such as through an iterative processes of learning in which the model adjusts and optimizes its parameters and algorithms to improve its performance on the designated task or series of tasks. This training phase may configure the model to develop the capability to perform its intended function with a high degree of accuracy and efficiency. In embodiments, the conditioning of the set of data may include modification, transformation, and/or the application of targeted algorithms to prepare the data for training. The conditioning step may be configured to ensure that the set of data is in an optimal state for training the model, resulting in an enhancement of the effectiveness of the model's learning process. These features and techniques not only qualify as patent-eligible features but also introduce substantial improvements to the field of computational modeling. These features are not merely theoretical but represent an integration of a concepts into practical applications that significantly enhance the functionality, reliability, and efficiency of the models developed through these processes.

In some embodiments, the disclosure includes techniques for generating a notification of an event (e.g., an output notification, a user notification, etc.) that includes generating an alert that includes information specifying the location of a source of data associated with the event (e.g., an ultrasonic generator, a water chiller, a centrifuge, etc.), formatting the alert into data structured according to an information format, and transmitting the formatted alert over a network to a device associated with a receiver based upon a destination address and a transmission schedule. In embodiments, receiving the alert enables a connection from the device associated with the receiver to the data source over the network when the device is connected to the source to retrieve the data associated with the event and causes a viewer application (e.g., a graphical user interface (GUI)) to be activated to display the data associated with the event. These features represent patent eligible features, as these features amount to significantly more than an abstract idea.

Such features, when considered as an ordered combination, amount to significantly more than simply organizing and comparing data. The features address the Internet-centric challenge of alerting a receiver with time sensitive information. This is addressed by transmitting the alert over a network to activate the viewer application, which enables the connection of the device of the receiver to the source over the network to retrieve the data associated with the event. These are meaningful limitations that add more than generally linking the use of an abstract idea (e.g., the general concept of organizing and comparing data) to the Internet, because they solve an Internet-centric problem with a solution that is necessarily rooted in computer technology. These features, when taken as an ordered combination, provide unconventional steps that confine the abstract idea to a particular useful application. Therefore, these features represent patent eligible subject matter.

Moreover, in embodiments, one or more operations and/or functionality of components described herein can be distributed across a plurality of computing systems (e.g., personal computers (PCs), user devices, servers, processors, etc.), such as by implementing the operations over a plurality of computing systems. This distribution can be configured to facilitate the optimal load balancing of requests, which can encompass a wide spectrum of network traffic or data transactions. By leveraging a distributed operational framework, a system implemented in accordance with embodiments of the present disclosure can effectively manage and mitigate potential bottlenecks, ensuring equitable processing distribution and preventing any single device from shouldering an excessive burden. This load balancing approach significantly enhances the overall responsiveness and efficiency of the network, markedly reducing the risk of system overload and ensuring continuous operational uptime. The technical advantages of this distributed load balancing can extend beyond mere efficiency improvements. It introduces a higher degree of fault tolerance within the network, where the failure of a single component does not precipitate a systemic collapse, markedly enhancing system reliability.

Additionally, this distributed configuration promotes a dynamic scalability feature, enabling the system to adapt to varying levels of demand without necessitating substantial infrastructural modifications. The integration of advanced algorithmic strategies for traffic distribution and resource allocation can further refine the load balancing process, ensuring that computational resources are utilized with optimal efficiency and that data flow is maintained at an optimal pace, regardless of the volume or complexity of the requests being processed. Moreover, the practical application of these disclosed features represents a significant technical improvement over traditional centralized systems. Through the integration of the disclosed technology into existing networks, entities can achieve a superior level of service quality, with minimized latency, increased throughput, and enhanced data integrity. The distributed approach of embodiments not only bolster the operational capacity of computing networks but offer a robust framework for the development of future technologies, underscoring its value as a foundational advancement in the field of network computing.

Further, to aid in the load balancing, the computing system can spawn multiple processes and threads to process data concurrently. The speed and efficiency of the computing system can be greatly improved by instantiating more than one process or thread to implement the claimed functionality. However, one skilled in the art of programming will appreciate that use of a single process or thread can also be utilized and is within the scope of the present disclosure.

Accordingly, the present disclosure discloses concepts inextricably tied to computer technology such that the present disclosure provides the technological benefit of implementing functionality to provide efficient and optimized generation of platelet lysate from PRP. The systems and techniques of embodiments provide improved systems by providing capabilities to perform functions that are currently performed manually and to perform functions that are currently not possible.

In one particular embodiment, a method for generating platelet lysate from PRP includes placing a predetermined volume of PRP into a centrifuge tube. The method further includes sealing the centrifuge tube and placing the sealed centrifuge tube containing the PRP into a water bath. The method further includes operating an ultrasonic generator to direct ultrasonic waves onto the PRP in the sealed centrifuge tube in the water bath for a predetermined amount of time, thereby generating platelet lysate from the PRP. The method further includes spinning the sealed centrifuge tube containing the platelet lysate in a centrifuge, thereby separating byproducts out of the platelet lysate.

In another embodiment, a system for generating platelet lysate from PRP includes one or more memory units and one or more computer processors. The one or more memory units are configured to store a plurality of platelet lysate parameters. The one or more computer processors are communicatively coupled to the one or more memory units. The one or more computer processors are configured to perform operations including accessing the plurality of platelet lysate parameters. The operations further include electronically communicating one or more first commands to a chilling system. The one or more first commands are operable to control the chilling system to chill a water bath to a predetermined temperature according to the plurality of platelet lysate parameters. The water bath includes a sealed centrifuge tube containing a predetermined volume of PRP. The operations further include electronically communicating one or more second commands to an ultrasonic generator. The one or more second commands are operable to control the ultrasonic generator to direct ultrasonic waves onto the PRP in the sealed centrifuge tube in the water bath for a predetermined amount of time according to the plurality of platelet lysate parameters, thereby generating platelet lysate from the PRP. The operations further include electronically communicating one or more third commands to a centrifuge. The one or more third commands are operable to control the centrifuge to spin the sealed centrifuge tube containing the platelet lysate in a centrifuge at a spin rate according to the plurality of platelet lysate parameters, thereby separating byproducts out of the platelet lysate.

In another embodiment, one or more computer-readable non-transitory storage media embodies instructions that, when executed by a processor, cause the processor to perform operations that include accessing a plurality of platelet lysate parameters stored in one or more memory units of a computer system. The operations further include electronically communicating one or more first commands to a chilling system. The one or more first commands are operable to control the chilling system to chill a water bath to a predetermined temperature according to the plurality of platelet lysate parameters. The water bath includes a sealed centrifuge tube containing a predetermined volume of PRP. The operations further include electronically communicating one or more second commands to an ultrasonic generator. The one or more second commands are operable to control the ultrasonic generator to direct ultrasonic waves onto the PRP in the sealed centrifuge tube in the water bath for a predetermined amount of time according to the plurality of platelet lysate parameters, thereby generating platelet lysate from the PRP. The operations further include electronically communicating one or more third commands to a centrifuge. The one or more third commands are operable to control the centrifuge to spin the sealed centrifuge tube containing the platelet lysate in a centrifuge at a spin rate according to the plurality of platelet lysate parameters, thereby separating byproducts out of the platelet lysate.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the disclosed embodiments and their features and advantages, reference is now made to the following description, taken in conjunction with the accompanying drawings, in which:

FIG. 1 illustrates a system for generating platelet lysate from platelet-rich plasma (PRP), according to particular embodiments;

FIG. 2 is flow chart of a method of generating platelet lysate from PRP, according to particular embodiments; and

FIG. 3 is an example computer system that can be utilized to implement aspects of the various technologies presented herein, according to particular embodiments.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DESCRIPTION OF EXAMPLE EMBODIMENTS

The disclosure presented in the following written description and the various features and advantageous details thereof, are explained more fully with reference to the non-limiting examples included in the accompanying drawings and as detailed in the description. Descriptions of well-known components have been omitted to not unnecessarily obscure the principal features described herein. The examples used in the following description are intended to facilitate an understanding of the ways in which the disclosure can be implemented and practiced. A person of ordinary skill in the art would read this disclosure to mean that any suitable combination of the functionality or exemplary embodiments below could be combined to achieve the subject matter claimed. The disclosure includes either a representative number of species falling within the scope of the genus or structural features common to the members of the genus so that one of ordinary skill in the art can recognize the members of the genus. Accordingly, these examples should not be construed as limiting the scope of the claims.

A person of ordinary skill in the art would understand that any system claims presented herein encompass all of the elements and limitations disclosed therein, and as such, require that each system claim be viewed as a whole. Any reasonably foreseeable items functionally related to the claims are also relevant. The Examiner, after having obtained a thorough understanding of the disclosure and claims of the present application has searched the prior art as disclosed in patents and other published documents, i.e., nonpatent literature. Therefore, as evidenced by issuance of this patent, the prior art fails to disclose or teach the elements and limitations presented in the claims as enabled by the specification and drawings, such that the presented claims are patentable under the applicable laws and rules of this jurisdiction.

Platelet lysate is the product of platelet activation to obtain the desired growth factors contained within the platelets. Platelet lysate may be used to stimulate tissue and wound healing, and to separate out the relatively inflammatory platelet cellular membrane proteins from the more clinically-desirable anti-inflammatory and growth-promoting proteins that facilitate healing. This release of platelet vesicles and granules has many uses in medicine, including stem cell culture media, tissue stimulation for healing, stimulation to promote nerve healing, scar tissue modification and reduction, and joint injection to reduce inflammation. The release of platelet vesicles and granules further promotes a more favorable environment for cartilage repair and enzymatic reduction of unhealthy tissue mass such as in spinal disk herniation, scar, and de-vascularized tissue that is associated with injury. Platelet lysate is rich in cytokines and growth factors and may be used to treat injured and degenerated connective tissues.

The field of interventional orthopedics is rapidly expanding with the growing recognition that healing of injured and degenerated connective tissues using naturally-derived growth factors is possible and proving to be safe and effective. Growth factors are becoming better categorized as they are discovered and have been found to stimulate healing via immunomodulatory effects, both pro-inflammatory and anti-inflammatory. Platelets in the circulation, when activated by an injury, naturally evoke the cascade of blood clotting, attract white blood cells to fight infection, attract macrophages to debride damaged tissue, attract circulating mesenchymal stem cells to exit the bloodstream and enter the local region, and stimulate fibroblasts and other local cells to rebuild as much as possible, the healthy tissue structure.

Autologous blood derived from the patient within a clinic setting can be minimally-processed using centrifugation to produce Platelet-Rich Plasma (PRP)—a mixture of proteins within the plasma and concentrated platelets and white blood cells. This can be further processed using techniques described herein to produce an acellular supernatant called platelet lysate and/or platelet releasate, which are rich in cytokines and growth factors and which clinically appear to be generally more anti-inflammatory than PRP.

Platelet lysate is typically used in applications such as wound healing, veterinary medicine, and ocular injury. Platelet lysate has also been proven to be helpful in cell culture media to accelerate the proliferation of cell types including mesenchymal stem cells, tenocytes, fibroblasts, chondrocytes, keratinocytes, and osteoblasts. It has proven to have advantages for human stem cell proliferation compared to the potential problems with using bovine serum, which had been used for cellular culture before platelet lysate became well-known.

Previously methods of producing platelet lysate include activation with calcium chloride, activation with fibrin, activation with thrombin, activation using ozone, activation using dextrose, and activation using a repeating sequence of freezing-thawing at very low temperatures of −80 C and below (e.g., using liquid nitrogen or a laboratory grade freezer than can reach the necessary temperatures). Each of these methods suffers from a variety of issues. The most important challenges are the time required, such as the freeze-thaw method which requires 6-12 hours or more and which requires equipment capable of reaching extremely cold temperatures. Furthermore, the use of added catalysts as the activation, which introduces variability, incompatibility, human-bovine immune incompatibility and viral vector risk, premature clot formation, and time-of-treatment constraints, will often cause the formation of a clot. This results in platelet lysate that is difficult or impossible to inject through a needle and requires the platelet lysate be used within minutes of production. Some methods require open exposure of the substrate to room air, requiring a laboratory-quality sterile hood environment for assurance of sterility and to reduce the risk of contamination. The performance of each of these methods has also been shown in studies to produce a yield of, at best, 40-60% of platelets activated.

To address these and other problems with previous techniques for producing platelet lysate, the disclosed embodiments utilize a water bath with a high-power ultrasonic transducer and coolant system to activate the platelets using a closed system which reduces and/or eliminates open air exposure, uses a simple but specific centrifuge tube, does not produce a clotting cascade, and takes less than twenty minutes of processing time. The disclosed embodiments provide a simpler procedure for producing platelet lysate from typical methods that can be fully accomplished in less than thirty minutes in an outpatient/clinic setting, eliminates the risk of air contamination, and eliminates exposure to exogenous, pathogenic, and non-human-derived factors. In addition, the yield of platelet lysate from the disclosed embodiments may approach 98-100% activation of the available platelets in the PRP solution, far exceeding exiting methods of platelet activation. As a result, the disclosed embodiments provide a simpler, safer, less inflammatory, and more time-efficient method of generating platelet lysate from PRP. The disclosed embodiments also produce a greatly improved yield of platelet activation over previous methods in order to produce platelet lysate for clinical use to heal damaged tissue, reduce inflammatory lesions, and stimulate healing of nerve injuries.

To efficiently and effectively generate platelet lysate from PRP, the present disclosure provides systems and methods of generating platelet lysate from PRP using ultrasonic waves. For example, certain embodiments provide methods that include placing a predetermined volume of PRP into a centrifuge tube, sealing the centrifuge tube, and placing the sealed centrifuge tube containing the PRP into a chilled water bath. The methods further include operating an ultrasonic generator to direct ultrasonic waves onto the PRP in the sealed centrifuge tube in the water bath for a predetermined amount of time, thereby generating platelet lysate from the PRP. The methods further include spinning the sealed centrifuge tube containing the platelet lysate in a centrifuge, thereby separating byproducts out of the platelet lysate. As a result, the disclosed embodiments are able to achieve dramatically higher yields of platelet lysate from typical processes-sometimes approaching around 99%.

In addition to being used in applications such as wound healing, veterinary medicine, and ocular injury, the platelet lysate produced by the systems and methods of the disclosed embodiments may be used to treat a wide variety of diseases and conditions. For example, the platelet lysate produced by the systems and methods of the disclosed embodiments may be used to treat strokes and other brain injuries. The platelet lysate produced by the systems and methods of the disclosed embodiments may also be used to treat degenerative nerve diseases such as Parkinson's disease and Multiple sclerosis (MS). Other diseases and conditions that the platelet lysate produced by the systems and methods of the disclosed embodiments may be used to treat include (but are not limited to): auto immune diseases; lupus; rheumatoid arthritis; eye diseases (e.g., macular degeneration); ENT diseases; diseases of the ear (e.g., Ménière's disease); pulmonary diseases; dental conditions (e.g., gingivitis); and the like.

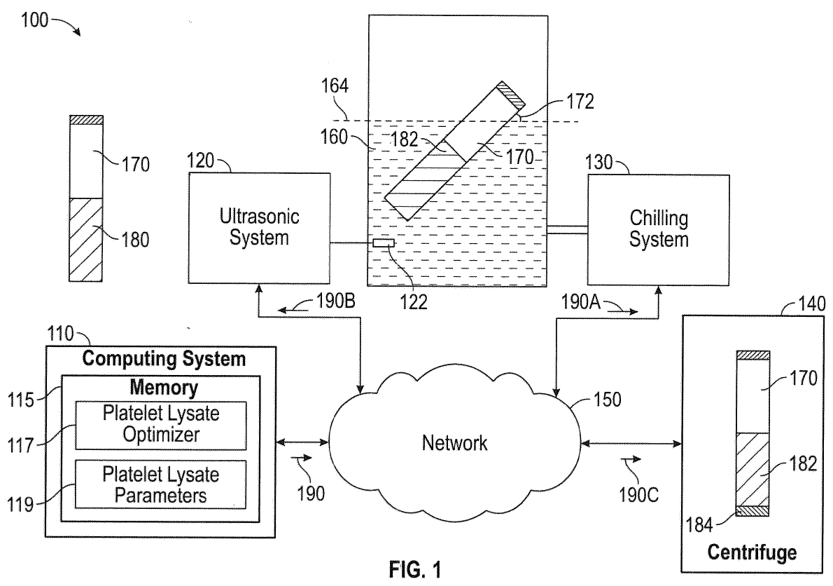

FIG. 1 illustrates a platelet lysate generating system 100 for effectively and efficiently generating platelet lysate from PRP, according to particular embodiments. In some embodiments, platelet lysate generating system 100 includes a computing system 110, an ultrasonic system 120, a water chilling system 130, a centrifuge 140, and a chilled water bath 160. In some embodiments, computing system 110, ultrasonic system 120, water chilling system 130, and centrifuge 140 may be communicatively coupled via a network 150. While FIG. 1 illustrates a particular embodiment of platelet lysate generating system 100, other embodiments of platelet lysate generating system 100 may have fewer or more components. For example, some embodiments of platelet lysate generating system 100 may not include a computing system 110 or a network 150.

In general, platelet lysate generating system 100 produces platelet lysate 182 from PRP 180. To do so, PRP 180 is extracted from a patient and placed into a centrifuge tube 170. Centrifuge tube 170 is then sealed and placed into chilled water bath 160. Computing system 110 or an operator commands water chilling system 130 to maintain chilled water bath 160 at a predetermined temperature (e.g., according to platelet lysate parameters 119 stored in memory 115). Next, computing system 110 or an operator commands ultrasonic system 120 to direct ultrasonic waves onto PRP 180 within sealed centrifuge tube 170 in chilled water bath 160, thereby generating platelet lysate 182 from PRP 180. Finally, computing system 110 or an operator commands centrifuge 140 to spin the sealed centrifuge tube 170 containing platelet lysate 182 in centrifuge 140, thereby separating byproducts 184 out of platelet lysate 182. As a result, platelet lysate 182 is quickly and efficiently produced from PRP 180.

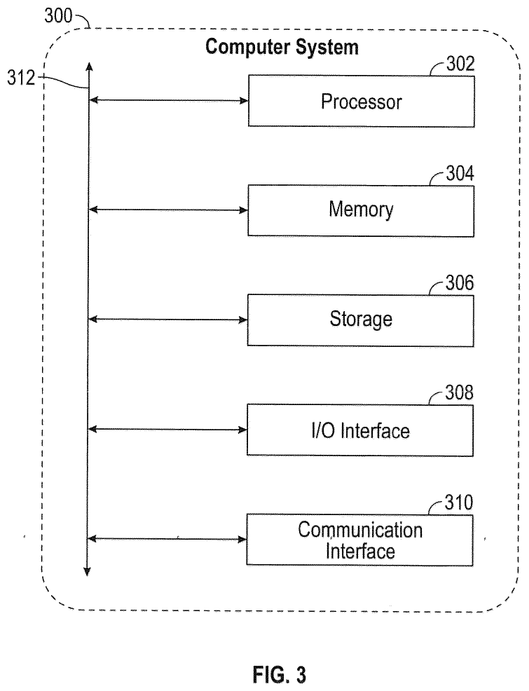

Computing system 110 may be any appropriate computing system in any suitable physical form. As example and not by way of limitation, computing system 110 may be an embedded computer system, a system-on-chip (SOC), a single-board computer system (SBC) (such as, for example, a computer-on-module (COM) or system-on-module (SOM)), a desktop computer system, a laptop or notebook computer system, a mainframe, a mesh of computer systems, a mobile telephone, a personal digital assistant (PDA), a server, a tablet computer system, an augmented/virtual reality device, or a combination of two or more of these. Where appropriate, computing system 110 may include one or more computer systems; be unitary or distributed; span multiple locations; span multiple machines; span multiple data centers; or reside in a cloud, which may include one or more cloud components in one or more networks. Where appropriate, computing system 110 may perform without substantial spatial or temporal limitation one or more steps of one or more methods described or illustrated herein. As an example, and not by way of limitation, computing system 110 may perform in real time or in batch mode one or more steps of one or more methods described or illustrated herein. Computing system 110 may perform at different times or at different locations one or more steps of one or more methods described or illustrated herein, where appropriate. A particular example of a computing system 110 is described in reference to FIG. 3.

Computing system 110 includes one or more memory units/devices 115 (collectively herein, “memory 115”) that may store platelet lysate optimizer 117 and platelet lysate parameters 119. Platelet lysate optimizer 117 may be a software module/application utilized by computing system 110 to generate platelet lysate 182 from PRP 180, as described herein. Platelet lysate optimizer 117 represents any suitable set of instructions, logic, or code embodied in a computer-readable storage medium. For example, platelet lysate optimizer 117 may be embodied in memory 115, a disk, a CD, or a flash drive. In particular embodiments, platelet lysate optimizer 117 may include instructions (e.g., a software application) executable by a computer processor to perform some or all of the functions described herein.

Platelet lysate parameters 119 include various settings for ultrasonic system 120, water chilling system 130, and centrifuge 140 that may be used by platelet lysate generating system 100 to efficiently and quickly generate platelet lysate 182 from PRP 180. For example, platelet lysate parameters 119 may include an amount of time and frequency for ultrasonic system 120, an amount of time and a temperature setting for water chilling system 130, and an amount of time and a spin rate setting (e.g., RPM) for centrifuge 140. In some embodiments, computing system 110 may access platelet lysate parameters 119 from memory 115 and send corresponding commands 190 (e.g., 190A-190C) to ultrasonic system 120, water chilling system 130, and centrifuge 140 either directly or via network 150. In some embodiments, platelet lysate parameters 119 may be displayed on an electronic display screen to a user.

Ultrasonic system 120 is any appropriate system for generating and transmitting ultrasonic energy/waves into PRP 180. In general, ultrasonic system 120 produces ultrasonic vibration energy that is directed onto PRP 180 within centrifuge tube 170 in chilled water bath 160 in order to generate platelet lysate 182 from PRP 180. In some embodiments, a user may directly control settings of ultrasonic system 120 (e.g., an amount of time, a percent amplitude, etc.). In other embodiments, computing system 110 may access platelet lysate parameters 119 and send one or more commands 190B to ultrasonic system 120 in order to control settings of ultrasonic system 120. In some embodiments, ultrasonic system 120 is QSonica Q500 Sonicator. In some embodiments, ultrasonic system 120 produces ultrasonic vibration energy with a titanium ultrasonic probe 122. In certain embodiments, ultrasonic probe 122 is mounted on a transducer that is passed through a flexible membrane that covers centrifuge tube 170 and is inserted directly into PRP 180. Ultrasonic probe 122 is vibrated within PRP 180 at ultrasonic speeds to lyse the platelets within PRP 180. This method, however, introduces the need for a flexible membrane to cover centrifuge tube 170. Undesirably, ultrasonic probe 122 may shed titanium ions and particulates into centrifuge tube 170 (e.g., into PRP 180). In addition, the tip of ultrasonic probe 122 must be autoclaved between uses. Ultrasonic probe 122 also may loosen from the transducer which requires frequent tightening during the process. Furthermore, this approach also causes some heat elevation within the specimen which needs to be monitored and controlled. To address these and other problems with ultrasonic probe 122 being inserted into centrifuge tube 170 and directly contacting PRP 180 within centrifuge tube 170, some embodiments utilize ultrasonic probe 122 that remains outside of centrifuge tube 170 (e.g., ultrasonic probe 122 does not directly contact PRP 180) as illustrated in FIG. 1. In these embodiments, centrifuge tube 170 remains completely sealed while in chilled water bath 160, and ultrasonic probe 122 remains within the water of chilled water bath 160. As a result, platelet lysate 182 may be produced from PRP 180 without contamination from ultrasonic probe 122 (e.g., shed titanium ions, heat, and the like).

Water chilling system 130 is any appropriate system for chilling and maintaining the water in chilled water bath 160 to a predetermined temperature. In some embodiments, a user may directly control settings of water chilling system 130 (e.g., an amount of time, a water temperature, etc.). In other embodiments, computing system 110 may access platelet lysate parameters 119 and send one or more commands 190A to water chilling system 130 in order to control settings of water chilling system 130. In some embodiments, water chilling system 130 is a QSonica ThermoCube that is used to chill the water within chilled water bath 160 in order to maintain a specimen temperature within physiological limits (e.g., between 5-14 degrees Celsius). Some embodiments may additionally use anti-pathogenic additives (e.g., Aqua Clear Water Conditioner at 2 ml per liter of water) in chilled water bath 160 to prevent contamination.

Centrifuge 140 is any appropriate centrifuge system for spinning centrifuge tube 170 containing platelet lysate 182. In some embodiments, a user may directly control settings of centrifuge 140 (e.g., an amount of time, a spin rate, etc.). In other embodiments, computing system 110 may access platelet lysate parameters 119 and send one or more commands 190C to centrifuge 140 in order to control settings of centrifuge 140.

Network 150 allows communication between and amongst the various components of platelet lysate generating system 100. This disclosure contemplates network 150 being any suitable network operable to facilitate communication between the components of platelet lysate generating system 100. Network 150 may include any interconnecting system capable of transmitting audio, video, signals, data, messages, or any combination of the preceding. Network 150 may include all or a portion of a local area network (LAN), a wide area network (WAN), an overlay network, a software-defined network (SDN), a virtual private network (VPN), a packet data network (e.g., the Internet), a mobile telephone network (e.g., cellular networks, such as 4G or 5G), a Plain Old Telephone (POT) network, a wireless data network (e.g., WiFi, WiGig, WiMax, etc.), a Long Term Evolution (LTE) network, a Universal Mobile Telecommunications System (UMTS) network, a peer-to-peer (P2P) network, a Bluetooth network, a Near Field Communication network, a Zigbee network, and/or any other suitable network.

Chilled water bath 160 is any appropriate container or system for holding centrifuge tube 170 at a certain position within water. In some embodiments, water from chilled water bath 160 is circulated through and chilled by water chilling system 130. In some embodiments, chilled water bath 160 includes one or more devices for holding centrifuge tube 170 at a specific position within the water of chilled water bath 160. For example, chilled water bath 160 may include an apparatus to hold centrifuge tube 170 in a position such that water line 164 is equal to or above PRP 180 within centrifuge tube 170 (e.g., all of PRP 180 is below water line 164). As another example, chilled water bath 160 may include an apparatus to hold centrifuge tube 170 at a specific angle 172 within chilled water bath 160 (e.g., an angle 172 of 45 degrees to water line 164). By holding centrifuge tube 170 at a specific position (e.g., such that all PRP 180 is below water line 164 and centrifuge tube 170 is held at a specific angle 172), the transfer of ultrasonic energy from ultrasonic probe 122 to PRP 180 may be optimized.

Centrifuge tube 170 is any appropriate container to hold PRP 180 and platelet lysate 182 within chilled water bath 160 and centrifuge 140. In some embodiments, centrifuge tube 170 is a 50 ml conical polypropylene centrifuge tube.

PRP 180 is PRP that has been extracted from a patient. In general, a starting volume of PRP 180 is placed into centrifuge tube 170 according to a desired end volume of platelet lysate 182. For example, if the desired amount of platelet lysate 182 is 5 mL, the starting volume of PRP 180 that is placed into centrifuge tube 170 may be 10 mL. As another example, if the desired amount of platelet lysate 182 is 7.5 mL, the starting volume of PRP 180 that is placed into centrifuge tube 170 may be 12.5 mL. As yet another example, if the desired amount of platelet lysate 182 is 10 mL, the starting volume of PRP 180 that is placed into centrifuge tube 170 may be 15 mL.

In operation, platelet lysate generating system 100 optimally produces platelet lysate 182 from PRP 180. To do so, PRP 180 is extracted from a patient and placed into a centrifuge tube 170. Centrifuge tube 170 is then sealed and placed into chilled water bath 160. In some embodiments, centrifuge tube 170 is placed at a specific angle 172 to water line 164 (e.g., 40-50 degrees). In some embodiments, centrifuge tube 170 is placed within chilled water bath 160 such that all PRP 180 is below water line 164.

Once centrifuge tube 170 with PRP 180 is placed within chilled water bath 160, computing system 110 (e.g., via one or more command 190A from platelet lysate optimizer 117) or an operator commands water chilling system 130 to maintain chilled water bath 160 at a predetermined temperature (e.g., according to platelet lysate parameters 119 stored in memory 115). For example, water chilling system 130 may be commanded to maintain the water within chilled water bath 160 to a temperature between 5-15 degrees Celsius, between 1-5 degrees Celsius, and the like.

Next, computing system 110 (e.g., via one or more command 190B from platelet lysate optimizer 117) or an operator commands ultrasonic system 120 to direct ultrasonic waves onto PRP 180 within sealed centrifuge tube 170 in chilled water bath 160, thereby generating platelet lysate 182 from PRP 180. In some embodiments, ultrasonic system 120 utilizes ultrasonic probe 122 within chilled water bath 160 to direct ultrasonic waves onto PRP 180 within centrifuge tube 170 (e.g., ultrasonic probe 122 does not directly contact PRP 180). In some embodiments, ultrasonic system 120 is commanded to operate for a predetermined amount of time (e.g., any amount of time between 15-30 minutes such as 25 minutes) and at a specific power level (e.g., 100% amplitude, 90-99% amplitude, 50-89% amplitude, etc.).

After ultrasonic system 120 is utilized to generate platelet lysate 182 from PRP 180, centrifuge tube 170 is placed in centrifuge 140 and then computing system 110 (e.g., via one or more command 190C from platelet lysate optimizer 117) or an operator commands centrifuge 140 to spin the sealed centrifuge tube 170 containing platelet lysate 182 in centrifuge 140, thereby separating byproducts 184 out of platelet lysate 182. For example, centrifuge 140 may be commanded to spin centrifuge tube 170 containing platelet lysate 182 for a specific amount of time (e.g., any amount of time between 1-10 minutes such as six minutes) at a specific spin rate (e.g., any number of RPMs between 2,000-5,000 RPMs such 4,000 RPMs). As a result, platelet lysate 182 is quickly and efficiently produced from PRP 180.

In some embodiments, platelet lysate 182 may be removed from centrifuge tube 170 after byproducts 184 are separated using centrifuge 140. Once removed, platelet lysate 182 may be passed through one or more filters to remove additional byproducts or contaminants. For example, platelet lysate 182 may be first passed through a 33 mm 45 μm PES filter. After being passed through the first filter, the filtered platelet lysate 182 may then be passed through a 33 mm 22 μm PES filter. This process may be repeated one or more times as desired.

In some embodiments, computing system 110 may send one or more electronic alerts (e.g., a text message and the like) to a user device (e.g., a smartphone, a computer, a tablet, etc.) to notify the user of the status of the various components of platelet lysate generating system 100. For example, computing system 110 may communicate with ultrasonic system 120 and determine that ultrasonic system 120 has completed the ultrasonic process to convert PRP 180 to platelet lysate 182. In response, computing system 110 may then send an alert or notification to the user device to notify the user of the completion of the ultrasonic process by ultrasonic system 120. As another example, computing system 110 may communicate with centrifuge 140 and determine that centrifuge 140 has completed the spinning process to separate byproducts 184 from platelet lysate 182. In response, computing system 110 may then send an alert or notification to the user device to notify the user of the completion of the spinning process by centrifuge 140. As a result, the efficiency of generating platelet lysate 182 from PRP 180 may be further improved.

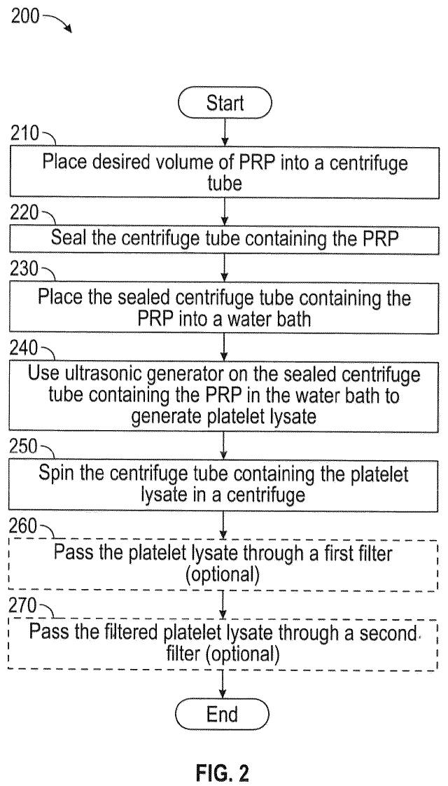

FIG. 2 is flow chart of a method 200 for generating platelet lysate 182 from PRP 180, according to particular embodiments. In some embodiments, method 200 may be performed by platelet lysate optimizer 117 of platelet lysate generating system 100 or an operator. At step 210, a desired volume of PRP is placed into a centrifuge tube. In some embodiments, the centrifuge tube is centrifuge tube 170. In some embodiments, the centrifuge tube is a conical polypropylene centrifuge tube.

At step 220, the centrifuge tube containing the PRP is sealed. At step 230, the sealed centrifuge tube containing the PRP is placed into a water bath. In some embodiments, the water bath is chilled water bath 160. In some embodiments, this step includes a computer system such as computing system 110 or an operator operating a water chilling system such as water chilling system 130 to chill the water within the water bath to a specific temperature (e.g., between 5 and 15 degrees Celsius). In some embodiments, the sealed centrifuge tube containing the PRP is placed into a water bath at a predetermined angle (e.g., between 40 and 50 degrees with respect to a water line of the water bath). In some embodiments, the sealed centrifuge tube containing the PRP is placed into a water bath such that all of the PRP is below the water line of the water bath.

At step 240, an ultrasonic generator is used by a computer system such as computing system 110 or an operator to direct ultrasonic waves onto the PRP in the sealed centrifuge tube in the water bath for a predetermined amount of time, thereby generating platelet lysate from the PRP. In some embodiments, the ultrasonic generator is ultrasonic system 120. In some embodiments, the ultrasonic generator utilizes an ultrasonic probe such as ultrasonic probe 122 within the water bath to direct ultrasonic waves onto the PRP within the sealed centrifuge tube (i.e., the ultrasonic probe does not directly contact the PRP). In some embodiments, the ultrasonic generator is commanded to operate for a predetermined amount of time (e.g., 25 minutes) and at a specific power level (e.g., 100% amplitude).

At step 250, a centrifuge is used by a computer system (e.g., computing system 110) or an operator to spin the sealed centrifuge tube containing the platelet lysate, thereby separating byproducts out of the platelet lysate. In some embodiments, the centrifuge is centrifuge 140. In some embodiments, the centrifuge is commanded to spin the centrifuge tube at a specific spin rate (e.g., 4,000 RPMs) for a specific amount of time (e.g., six minutes). After the byproducts are separated from the platelet lysate, the platelet lysate may be removed from the centrifuge tube and administered to the patient. After step 250, method 200 may end.

At step 260, which may be an optional step, the platelet lysate from step 250 is passed through a first filter. In some embodiments, the first filter is a 45 μm filter. At step 270, which may also be an optional step, the filtered platelet lysate from step 260 may be passed through a second filter. In some embodiments, the second filter is a 22 μm filter. Additional filtering steps such as steps 260 and 270 may be utilized as desired.

Particular embodiments may repeat one or more steps of the method of FIG. 2, where appropriate. Although this disclosure describes and illustrates particular steps of the method of FIG. 2 as occurring in a particular order, this disclosure contemplates any suitable steps of the method of FIG. 2 occurring in any suitable order. Moreover, although this disclosure describes and illustrates an example method including the particular steps of the method of FIG. 2, this disclosure contemplates any suitable method including any suitable steps, which may include all, some, or none of the steps of the method of FIG. 2, where appropriate. Furthermore, although this disclosure describes and illustrates particular components, devices, or systems carrying out particular steps of the method of FIG. 2, this disclosure contemplates any suitable combination of any suitable components, devices, or systems carrying out any suitable steps of the method of FIG. 2.

FIG. 3 illustrates an example computer system 300 that can be utilized to implement aspects of the various methods and systems presented herein, according to particular embodiments. In particular embodiments, one or more computer systems 300 perform one or more steps of one or more methods described or illustrated herein. In particular embodiments, one or more computer systems 300 provide functionality described or illustrated herein. In particular embodiments, software running on one or more computer systems 300 performs one or more steps of one or more methods described or illustrated herein or provides functionality described or illustrated herein. Particular embodiments include one or more portions of one or more computer systems 300. Herein, reference to a computer system may encompass a computing device, and vice versa, where appropriate. Moreover, reference to a computer system may encompass one or more computer systems, where appropriate.

This disclosure contemplates any suitable number of computer systems 300. This disclosure contemplates computer system 300 taking any suitable physical form. As example and not by way of limitation, computer system 300 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 300 may include one or more computer systems 300; be unitary or distributed; span multiple locations; span multiple machines; span multiple data centers; or reside in a cloud, which may include one or more cloud components in one or more networks. Where appropriate, one or more computer systems 300 may perform without substantial spatial or temporal limitation one or more steps of one or more methods described or illustrated herein. As an example, and not by way of limitation, one or more computer systems 300 may perform in real time or in batch mode one or more steps of one or more methods described or illustrated herein. One or more computer systems 300 may perform at different times or at different locations one or more steps of one or more methods described or illustrated herein, where appropriate.

In particular embodiments, computer system 300 includes a processor 302, memory 304, storage 306, an input/output (I/O) interface 308, a communication interface 310, and a bus 312. Although this disclosure describes and illustrates a particular computer system having a particular number of particular components in a particular arrangement, this disclosure contemplates any suitable computer system having any suitable number of any suitable components in any suitable arrangement.

In particular embodiments, processor 302 includes hardware for executing instructions, such as those making up a computer program. As an example, and not by way of limitation, to execute instructions, processor 302 may retrieve (or fetch) the instructions from an internal register, an internal cache, memory 304, or storage 306; decode and execute them; and then write one or more results to an internal register, an internal cache, memory 304, or storage 306. In particular embodiments, processor 302 may include one or more internal caches for data, instructions, or addresses. This disclosure contemplates processor 302 including any suitable number of any suitable internal caches, where appropriate. As an example, and not by way of limitation, processor 302 may include one or more instruction caches, one or more data caches, and one or more translation lookaside buffers (TLBs). Instructions in the instruction caches may be copies of instructions in memory 304 or storage 306, and the instruction caches may speed up retrieval of those instructions by processor 302. Data in the data caches may be copies of data in memory 304 or storage 306 for instructions executing at processor 302 to operate on; the results of previous instructions executed at processor 302 for access by subsequent instructions executing at processor 302 or for writing to memory 304 or storage 306; or other suitable data. The data caches may speed up read or write operations by processor 302. The TLBs may speed up virtual-address translation for processor 302. In particular embodiments, processor 302 may include one or more internal registers for data, instructions, or addresses. This disclosure contemplates processor 302 including any suitable number of any suitable internal registers, where appropriate. Where appropriate, processor 302 may include one or more arithmetic logic units (ALUs); be a multi-core processor; or include one or more processors 302. Although this disclosure describes and illustrates a particular processor, this disclosure contemplates any suitable processor.

In particular embodiments, memory 304 includes main memory for storing instructions for processor 302 to execute or data on which processor 302 may operate. As an example, and not by way of limitation, computer system 300 may load instructions from storage 306 or another source (such as, for example, another computer system 300) to memory 304. Processor 302 may then load the instructions from memory 304 to an internal register or internal cache. To execute the instructions, processor 302 may retrieve the instructions from the internal register or internal cache and decode them. During or after execution of the instructions, processor 302 may write one or more results (which may be intermediate or final results) to the internal register or internal cache. Processor 302 may then write one or more of those results to memory 304. In particular embodiments, processor 302 executes only instructions in one or more internal registers or internal caches or in memory 304 (as opposed to storage 306 or elsewhere) and operates only on data in one or more internal registers or internal caches or in memory 304 (as opposed to storage 306 or elsewhere). One or more memory buses (which may each include an address bus and a data bus) may couple processor 302 to memory 304. Bus 312 may include one or more memory buses, as described below. In particular embodiments, one or more memory management units (MMUs) reside between processor 302 and memory 304 and facilitate accesses to memory 304 requested by processor 302. In particular embodiments, memory 304 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 304 may include one or more memories 304, where appropriate. Although this disclosure describes and illustrates particular memory, this disclosure contemplates any suitable memory.

In particular embodiments, storage 306 includes mass storage for data or instructions. As an example, and not by way of limitation, storage 306 may include a hard disk drive (HDD), a floppy disk drive, flash memory, an optical disc, a magneto-optical disc, magnetic tape, or a Universal Serial Bus (USB) drive or a combination of two or more of these. Storage 306 may include removable or non-removable (or fixed) media, where appropriate. Storage 306 may be internal or external to computer system 300, where appropriate. In particular embodiments, storage 306 is non-volatile, solid-state memory. In particular embodiments, storage 306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 306 taking any suitable physical form. Storage 306 may include one or more storage control units facilitating communication between processor 302 and storage 306, where appropriate. Where appropriate, storage 306 may include one or more storages 306. Although this disclosure describes and illustrates particular storage, this disclosure contemplates any suitable storage.

In particular embodiments, I/O interface 308 includes hardware, software, or both, providing one or more interfaces for communication between computer system 300 and one or more I/O devices. Computer system 300 may include one or more of these I/O devices, where appropriate. One or more of these I/O devices may enable communication between a person and computer system 300. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 308 for them. Where appropriate, I/O interface 308 may include one or more device or software drivers enabling processor 302 to drive one or more of these I/O devices. I/O interface 308 may include one or more I/O interfaces 308, where appropriate. Although this disclosure describes and illustrates a particular I/O interface, this disclosure contemplates any suitable I/O interface.

In particular embodiments, communication interface 310 includes hardware, software, or both providing one or more interfaces for communication (such as, for example, packet-based communication) between computer system 300 and one or more other computer systems 300 or one or more networks. As an example, and not by way of limitation, communication interface 310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 310 for it. As an example, and not by way of limitation, computer system 300 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 300 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network, a Long-Term Evolution (LTE) network, or a 5G network), or other suitable wireless network or a combination of two or more of these. Computer system 300 may include any suitable communication interface 310 for any of these networks, where appropriate. Communication interface 310 may include one or more communication interfaces 310, where appropriate. Although this disclosure describes and illustrates a particular communication interface, this disclosure contemplates any suitable communication interface.

In particular embodiments, bus 312 includes hardware, software, or both coupling components of computer system 300 to each other. As an example and not by way of limitation, bus 312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 312 may include one or more buses 312, where appropriate. Although this disclosure describes and illustrates a particular bus, this disclosure contemplates any suitable bus or interconnect.

Although the present disclosure and its advantages have been described in detail, it should be understood that various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

Moreover, the description in this patent document should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “member,” “module,” “device,” “unit,” “component,” “element,” “mechanism,” “apparatus,” “machine,” “system,” “processor,” “processing device,” or “controller” within a claim can be understood and intended to refer to structures known to those skilled in the relevant art, as further modified or enhanced by the features of the claims themselves, and can be not intended to invoke 35 U.S.C. § 112(f). Even under the broadest reasonable interpretation, in light of this paragraph of this specification, the claims are not intended to invoke 35 U.S.C. § 112(f) absent the specific language described above.

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the disclosures can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

1. A method for generating platelet lysate from platelet-rich plasma (PRP), the method comprising:
placing a predetermined volume of PRP into a centrifuge tube;
sealing the centrifuge tube;
placing the sealed centrifuge tube containing the PRP into a water bath;
operating an ultrasonic generator to direct ultrasonic waves onto the PRP in the sealed centrifuge tube in the water bath for a predetermined amount of time, thereby generating platelet lysate from the PRP; and
spinning the sealed centrifuge tube containing the platelet lysate in a centrifuge, thereby separating byproducts out of the platelet lysate.

2. The method of claim 1, further comprising:
adjusting the sealed centrifuge tube containing the PRP to a predetermined angle in the water bath prior to directing ultrasonic waves onto the PRP in the sealed centrifuge tube.

3. The method of claim 2, wherein the predetermined angle comprises an angle that is between 40 and 50 degrees with respect to a water line of the water bath.

4. The method of claim 1, further comprising:
operating a chilling system to cool the water bath to a predetermined temperature prior to directing ultrasonic waves onto the PRP in the sealed centrifuge tube.

5. The method of claim 4, wherein the predetermined temperature is between 5 and 15 degrees Celsius.

6. The method of claim 1, further comprising:
after spinning the sealed centrifuge tube containing the platelet lysate in the centrifuge, passing the platelet lysate through a first filter to generate filtered platelet lysate.

7. The method of claim 6, further comprising:
after passing the platelet lysate through the first filter to generate filtered platelet lysate, passing the filtered platelet lysate through a second filter.

8. The method of claim 1, wherein the ultrasonic generator directs ultrasonic waves onto the PRP in the sealed centrifuge tube in the water bath using an ultrasonic probe.

9. The method of claim 8, wherein the ultrasonic probe does not contact the PRP within the sealed centrifuge tube.

10. A system for generating platelet lysate from platelet-rich plasma (PRP), the system comprising:
one or more memory units configured to store a plurality of platelet lysate parameters; and
one or more computer processors communicatively coupled to the one or more memory units and configured to perform operations comprising:
accessing the plurality of platelet lysate parameters;
electronically communicating one or more first commands to a chilling system, the one or more first commands operable to control the chilling system to chill a water bath to a predetermined temperature according to the plurality of platelet lysate parameters, the water bath comprising a sealed centrifuge tube containing a predetermined volume of PRP;
electronically communicating one or more second commands to an ultrasonic generator, the one or more second commands operable to control the ultrasonic generator to direct ultrasonic waves onto the PRP in the sealed centrifuge tube in the water bath for a predetermined amount of time according to the plurality of platelet lysate parameters, thereby generating platelet lysate from the PRP; and
electronically communicating one or more third commands to a centrifuge, the one or more third commands operable to control the centrifuge to spin the sealed centrifuge tube containing the platelet lysate in a centrifuge at a spin rate according to the plurality of platelet lysate parameters, thereby separating byproducts out of the platelet lysate.

11. The system of claim 10, wherein the sealed centrifuge tube containing the PRP is adjusted to a predetermined angle in the water bath prior to the ultrasonic generator directing ultrasonic waves onto the PRP in the sealed centrifuge tube.

12. The system of claim 11, wherein the predetermined angle comprises an angle that is between 40 and 50 degrees with respect to a water line of the water bath.

13. The system of claim 10, wherein the predetermined temperature is between 5 and 15 degrees Celsius.

14. The system of claim 10, wherein the ultrasonic generator directs ultrasonic waves onto the PRP in the sealed centrifuge tube in the water bath using an ultrasonic probe.

15. The system of claim 14, wherein the ultrasonic probe does not contact the PRP within the sealed centrifuge tube.

16. One or more computer-readable non-transitory storage media embodying instructions that, when executed by a processor, cause the processor to perform operations comprising:
accessing a plurality of platelet lysate parameters stored in one or more memory units of a computer system;
electronically communicating one or more first commands to a chilling system, the one or more first commands operable to control the chilling system to chill a water bath to a predetermined temperature according to the plurality of platelet lysate parameters, the water bath comprising a sealed centrifuge tube containing a predetermined volume of platelet-rich plasma (PRP);
electronically communicating one or more second commands to an ultrasonic generator, the one or more second commands operable to control the ultrasonic generator to direct ultrasonic waves onto the PRP in the sealed centrifuge tube in the water bath for a predetermined amount of time according to the plurality of platelet lysate parameters, thereby generating platelet lysate from the PRP; and
electronically communicating one or more third commands to a centrifuge, the one or more third commands operable to control the centrifuge to spin the sealed centrifuge tube containing the platelet lysate in a centrifuge at a spin rate according to the plurality of platelet lysate parameters, thereby separating byproducts out of the platelet lysate.

17. The one or more computer-readable non-transitory storage media of claim 16, wherein the sealed centrifuge tube containing the PRP is adjusted to a predetermined angle in the water bath prior to the ultrasonic generator directing ultrasonic waves onto the PRP in the sealed centrifuge tube.

18. The one or more computer-readable non-transitory storage media of claim 17, wherein the predetermined angle comprises an angle that is between 40 and 50 degrees with respect to a water line of the water bath.

19. The one or more computer-readable non-transitory storage media of claim 16, wherein the predetermined temperature is between 5 and 15 degrees Celsius.

20. The one or more computer-readable non-transitory storage media of claim 16, wherein:
the ultrasonic generator directs ultrasonic waves onto the PRP in the sealed centrifuge tube in the water bath using an ultrasonic probe; and
the ultrasonic probe does not contact the PRP within the sealed centrifuge tube.
